SEQUENCE LISTING (8 sequences)

SEQ ID NO: 1 - DNA, 20 bp, Artificial Sequence
  probe
tttttttttttttttttttt

SEQ ID NO: 2 - DNA, 26 bp, Artificial Sequence
  probe
tttttttttttttttttttttttttt

SEQ ID NO: 3 - DNA, 30 bp, Artificial Sequence
  probe
tttttttttttttttttttttttttttttt

SEQ ID NO: 4 - DNA, 35 bp, Artificial Sequence
  probe
ttttttttttttttttttttttttttttttttttt

SEQ ID NO: 5 - DNA, 40 bp, Artificial Sequence
  probe
tttttttttttttttttttttttttttttttttttttttt

SEQ ID NO: 6 - DNA, 45 bp, Artificial Sequence
  probe
ttttttttttttttttttttttttttttttttttttttttttttt

SEQ ID NO: 7 - DNA, 25 bp, Artificial Sequence
  marker
ccccccccccccccccccccccccc

SEQ ID NO: 8 - DNA, 45 bp, Artificial Sequence
  target DNA
aaaaaaaaaaaaaaaaaaaaaaaaaaaaaaaaaaaaaaaaaaaaa

(19) United States
(12) Patent Application Publication
(10) Pub. No.: US 2007/0003958 A1
(43) Pub. Date: Jan. 4, 2007

(54) Method for measuring melting temperature of nucleic acid hybrid and apparatus for use in the method

(75) Inventors: Tadashi Okamoto, Yokohama-shi (JP); Yuri Mizutani, Yokohama-shi (JP)

Correspondence Address: FITZPATRICK CELLA HARPER & SCINTO, 30 ROCKEFELLER PLAZA, NEW YORK, NY, 10112, US

(73) Assignee: Canon Kabushiki Kaisha, Tokyo (JP)

(21) Appl. No.: 11/473,061

(22) Filed: Jun. 23, 2006

(30) Foreign Application Priority Data
Jun. 29, 2005 (JP) 2005-189840

(51) Int. Cl. C12Q 1/68; G01N 21/00; C12M 1/34

(52) U.S. Cl. 435/6; 435/287.2; 977/924; 356/73

(57) ABSTRACT

The melting temperature of a hybrid formed of a first nucleic acid, such as a probe, immobilized on a surface of a substrate and a second nucleic acid hybridizable with the first nucleic acid is measured by detecting formation or dissociation of the hybrid depending upon temperature by an optical system focused on the surface of the substrate based on an intensity of fluorescence derived from the second nucleic acid that is fluorescence-labeled.

### FIELD OF THE INVENTION

The present invention relates to a method for measuring the melting temperature of a nucleic acid hybrid on a solid phase substrate and an apparatus for use in the method.

### BACKGROUND OF THE INVENTION

A DNA probe method has been widely used to detect a gene since the “Southern blotting” era. Basically, in the DNA probe method, nucleic acids such as DNA and RNA having mutually complementary base sequences form double strands (hybrid) such that the double strands recognize each other.

In recent years, a DNA chip has been developed for detecting multiple types of genes at the same time. The principle of the DNA chip resides in hybridization. Also, sequencing or priming used in polymerase chain reaction (PCR) (in strict sense, they may not be included in the category of gene detection) is based on hybridization.

There are conditions for successfully performing hybridization. Once hybridized, the intensity of hybridization (stability of double strands) is influenced by certain conditions. These conditions are summarized below.

(1) To successfully perform hybridization, the entire base sequence of the part of two nucleic acid chains (triple and quadruple chains are not discussed herein) at which hybridization takes place must be basically complementary (in full match or perfect match).

(2) When a non-complementary base (mismatch) is present in the base sequence of the part at which hybridization takes place, a double strand may not be formed, or even if a double strand is formed, the stability of the double strand basically decreases. The stability decreases as the base number of such a mismatch portion increases.

(3) Depending upon the number and position of mismatch bases, a higher-order structure, such as a bulge and a loop, may occur in the double strand after formed.

(4) Basically, as the number of bases (chain length) of a double strand increases, the stability of the double strand increases.

(5) As the ratio of base pairs of guanine (G)/cytosine (C) increases, the stability of the double strand basically increases. Conversely, as the ratio of base pairs of adenine (A)/thymine (T) (adenine (A)/uracil (U) in the case of RNA) increases, the stability of the double bond decreases. This is because the number of hydrogen bonds forming a base pair differs between them.

(6) Even if double strands have the same chain length and the same base pair ratio, the stability of them sometimes differs depending upon a positional distribution of the base pairs in the chain.

(7) Since hybridization is mediated by a hydrogen bond, the stability of a double strand decreases with an increase of the temperature of a solution dissolving the double strand.

(8) When pH of the solution changes, the stability of a double strand may sometimes vary. This is because individual bases intrinsically have a plurality of acid dissociation constants (pKa).

(9) The stability of a double strand changes depending upon the salt concentration of a solution dissolving the double strand. This is because the negative charge repulsion between phosphates of individual nucleic acid chains is suppressed by positive ions (cations) derived from a salt.

(10) When any one of the nucleic acid chains or each of them forms a higher-order structure, with the result that the stability of individual nucleic acid chains changes, the stability of a double strand or a double strand formation speed may change in some cases.

As described above, the stability of a nucleic acid double strand varies depending upon various conditions. As an indicator for evaluating such stability, the melting temperature (Tm) of a double-stranded nucleic acid is known.

Now, a melting temperature and a method of measuring the same of a double-stranded nucleic acid formed of single-stranded nucleic acids that can be hybridized with each other in a liquid phase will be schematically described below, taking DNA as an example.

Two DNA chains having mutually complementary base sequences recognize each other in optimum conditions to form a double strand, which is known as a Watson-Crick double-stranded helical structure. Within the double helical structure, a base pair is formed via a hydrogen bond, which is formed within a plane (virtually slightly inclined). These base pairs are continuously (repeatedly) formed along the helical while varying the angle of the hydrogen bond little by little. Thus, individual base pairs take a stacking structure where one base pair is on top of the other. As a result, the electron cloud of a π electron overlaps with those of adjacent π electrons positioned above and below, stabilizing the state of electrons. A nucleic acid base has a light absorbance at a wavelength near 260 nm. However, the intensity of the absorption decreases when DNA changes from a single strand to a double strand, due to the stabilization effect produced by the stacking structure. Therefore, based on the absorbance change, the process of changing a single strand to a double strand, and vice versa, can be monitored.

As already described above, the stability of the double-stranded nucleic acid chain varies depending upon the temperature. The process (from a single strand to a double strand, and vice versa) is generally monitored by gradually increasing or decreasing the temperature of a nucleic acid solution and measuring the absorbance of the resultant solution. At this time, a single-stranded nucleic acid abruptly changes into a double-stranded nucleic acid, and vice versa upon reaching a specific temperature. The specific temperature is called the melting temperature of the nucleic acid.

The double strand formed of nucleic acids in combination having a high melting temperature has a high stability, whereas that formed of nucleic acids in combination having a low melting temperature has a low stability.

In methods using formation of a double-stranded nucleic acid, such as a DNA probe method, nucleic acid sequencing method, and PCR method, the stability of a double-stranded nucleic acid is an extremely important factor because desired results cannot be obtained if a desired hybrid is not generated.

Therefore, when the aforementioned methods are used, it is a common practice to estimate the stability of a hybrid of nucleic acids to be employed in advance. As a method of estimating the stability of a hybrid, it is known that the melting temperature of the hybrid is calculated using the stability of base pairs, A:T pair and G:C pair, as standard values in consideration of other parameters such as chain length, G:C content, and tendency of a single-stranded DNA chain to form a higher-order structure. As another estimation method, it is known that a melting temperature is estimated from the stability parameter of nearest neighbor base pair experimentally obtained in consideration of a base sequence (Biotechniques Vol. 27, 1218-1228, 1999).

As described above, the melting temperature of a nucleic acid can be computationally estimated to some extent based on parameters; however, computational methods are not always perfect. In some cases, an estimated melting temperature differs from an actual melting temperature by the presence of an unpredictable factor.

Accordingly, to know the melting temperature of a nucleic acid accurately, first the absorbance of a solution actually containing the nucleic acid is measured while changing the temperature of the solution, as described above, to obtain a profile. The melting temperature of the nucleic acid can be obtained from the profile. To describe more specifically, absorbance is measured while changing the temperature of a nucleic acid solution (several ml) at a rate of 0.2° C. to 0.5° C./minute within the temperature range of 60° C. to 80° C. When the melting temperature is measured in this manner, it may take 6 to 8 hours for a single measurement. Lately, an apparatus, DU 640, manufactured by Beckman Coulter, Inc., which can measure 6 types of nucleic acid solutions (about 600 μl each) at the same time, has come onto the market. Therefore, if such an apparatus is used, the measurement efficiency can be improved. However, even in either case, an enormous amount of time is required to measure a wide variety of nucleic acids.

A conventional technique for measuring a melting temperature is based on capturing a slight difference in absorbance. Therefore, it is principally difficult to detect the melting temperature of a hybrid between a probe of several tens of bases and a target nucleic acid of several hundreds or several thousands of bases practically used in gene analysis. Even if the melting temperature of such a hybrid can be measured, the measurement presumably involves extremely significant errors.

On the other hand, various methods including Southern blotting have been developed for detecting target nucleic acids in the beginning of the 1990s. Of them, in a method called a nucleic acid chip where a large number of nucleic acid probes are arranged in the matrix form and fixed on a solid phase substrate, the nucleic acid probes fixed on a solid phase substrate are reacted with nucleic acid samples to detect a target nucleic acid. In this method, hybridization is performed in the solid/liquid interface (called solid-phase hybridization). In the solid-phase hybridization, the stability of a hybrid, in other words, the melting temperature of a hybrid, is presumably changed by various factors including the state of a substrate surface, the interaction between a probe or a target nucleic acid and a substrate surface, the adhesiveness of a probe or a target nucleic acid to a substrate surface, the chain length of a probe or a target nucleic acid, the position of a complementary base sequence in the entire length of each of a probe and a target nucleic acid, the position of a labeling substance in a probe or a target nucleic acid, the interaction between a labeling substance and a substrate surface, and a decrease in the collision number of a probe and a target nucleic acid. Therefore, it is difficult to discuss the melting temperature of solid phase hybridization directly with reference to the melting temperature of liquid phase hybridization calculated or measured. In these circumstances, it has been required to develop an efficient method of measuring the melting temperatures of a plurality of types of hybrids obtained by solid phase hybridization.

It is conceivable that the melting temperature may be obtained based on a profile of absorbance changing with temperature as is applied in the hybridization in a liquid phase. However, such a measurement method cannot be used in practice because the nucleic acid present on the surface of a solid phase is of at most 2 molecules, exhibiting an extremely small absorbance.

Then, as a method of measuring the melting temperatures of a plurality of types of nucleic acids, a dynamic allele specific hybridization (DASH) has been developed by ThermoHybaid Limited, UK. In this method, of a double-stranded nucleic acid, only the single strand that is desired to be analyzed is subjected to PCR using a primer labeled with biotin. The PCR product is immobilized on a microplate previously coated with streptavidin, and denatured by alkali. In this way, only the single strand of the double-stranded nucleic acid that is desired to be analyzed is left on the microplate. In a next step, an oligo nucleic acid probe is added to form a hybrid on the plate. Subsequently, a fluorescent dye such as SYBR Green I (Molecular Probe Inc. USA), which intercalates into a double-stranded nucleic acid to emit fluorescence, is reacted to the hybrid. Thereafter, when the temperature of the microplate in this state is changed, the melting temperature of the double-stranded nucleic acid formed in each of the wells of the plate is obtained based on a change in fluorescent amount emitted from the fluorescent dye.

### SUMMARY OF THE INVENTION

According to the DASH method, it is possible to measure the melting temperatures of several hundreds types of double-stranded nucleic acids at the same time, if necessary. However, the DASH method has the following problems.

Generally, hybridization analysis based on fluorescence is performed by labeling a target nucleic acid with a fluorescent dye (generally via a covalent bond) and measuring the intensity of fluorescence emitted from the fluorescent dye. However, it is difficult to apply this method to the DASH method because a fluorescence-labeled target nucleic acid remains unhybridized in a well of the microplate. The fluorescence emitted from the non-hybridized nucleic acid is overwhelmingly strong, so that it is difficult to correctly measure the fluorescence emitted from the target hybridized nucleic acid. When the melting temperature of a hybrid is measured while increasing the temperature, the double-stranded nucleic acids of the hybrid are dissociated and a target nucleic acid is dispersed into a solution. Conversely, when the melting temperature of a hybrid is measured while decreasing the temperature, since a target nucleic acid to be hybridized must be dissolved in the solution, the unhybridized target nucleic acid cannot be washed away in principal. Therefore, it is basically impossible to eliminate an effect of fluorescence emitted from the fluorescence-labeled target nucleic acid remaining unhybridized in a well, in measuring the melting temperature.

Because of such a problem, the DASH method employs an intercalator dye. However, an intercalator may intercalate into a target nucleic acid by recognizing a different number of molecules depending upon the chain length and the base sequence of a target nucleic acid. Therefore, when a melting temperature is measured, the fluorescence intensity may not be stably obtained. It is also known that when an intercalator dye may nonspecifically be adsorbed onto the surface of a substrate made of plastic and glass, etc. or the surface of such a substrate treated (coated) with an organic compound, may emit fluorescence in some cases. In these cases, fluorescence cannot be obtained in a stable amount or fails to correctly reflect the amount of hybrids. As a matter of fact, SYBR Green I, which is a dye relatively difficult to be adsorbed onto a plastic surface, is sometimes adsorbed nonspecifically to a glass surface or a surface-treated coated glass surface. In addition, an intercalator has an intrinsic problem such that it may emit fluorescence more or less even if it does not intercalate. Furthermore, the fluorescence intensity of an intercalator is strongly affected by a temperature change, as compared to a general fluorescent dye, which is also a characteristic problem of the intercalator. When such an intercalator is used in measuring a melting temperature while changing the temperature in a relatively broad range, the fluorescence intensity varies depending upon temperature. As a result, it becomes difficult to measure a melting temperature correctly. In addition to such a basic problem, when an intercalator intercalates into a nucleic acid hybrid, it affects the stability of the nucleic acid hybrid itself. In this case, even if the melting temperature can be measured, the measured melting temperature becomes essentially meaningless.

Furthermore, when fluorescence emitted from a fluorescent dye present in wells is observed under a fluorescence microscope and the intensity of the fluorescence is measured, it is advantageous if the bottom surface is uniformly flat within each well. This is because it is not required to adjust the focus of the fluorescence microscope, eliminating complicated focus adjustment. Furthermore, if the flatness of the bottom surface does not differ between wells, it is not required to adjust the focus of the fluorescence microscope between wells. Furthermore, if the flatness of the bottom surface of a well is not adequate, and if the flatness of the bottom surface differs between wells, a correct amount of fluorescence may not be obtained by the presence of various factors such as the type of fluorescence detection system, the position for observing fluorescence, and the shape and size of a well.

When a melting temperature is measured by using a microplate, about several hundreds, several thousands to several tens of thousands of hybrids can be measured; however, in consideration of the structure of the microplate, it is actually impossible to measure the melting temperatures of hybrids exceeding several tens of thousands.

Furthermore, as described above, the melting temperature of a double-stranded nucleic acid varies depending upon the state of the substrate surface. Therefore, when a probe is immobilized on a flat substrate having no well, as is in the case of a nucleic acid chip, the melting temperature may differ from that measured by the DASH method.

An object of the present invention is to provide a method of measuring a melting temperature, which enables to measure the melting temperature of a double-stranded nucleic acid bound to a substrate and the melting temperatures of individual double-stranded nucleic acids hybridized with corresponding probes immobilized on a nucleic acid chip, and an apparatus for use in the method.

The present invention is attained to solve the above problems of conventional art.

According to a first aspect of the present invention, there is provided a method of measuring a melting temperature of a double-stranded nucleic acid comprising the steps of:

(1) supplying a second nucleic acid having a base sequence hybridizable with a first nucleic acid to a substrate having the first nucleic acid immobilized thereon to form a reaction system;

(2) monitoring a change in a reaction by changing a temperature of the reaction system; and

(3) determining a melting temperature of a hybrid formed on the substrate based on a profile of the reaction monitored.

According to a second aspect of the present invention, there is provided a method of measuring a melting temperature of a double-stranded nucleic acid comprising the steps of:

(1) preparing a nucleic acid chip (also called nucleic acid array) in which a plurality of types of first nucleic acids are separately immobilized on a surface of a substrate partitioned into immobilizing regions;

(2) preparing a plurality of types of second nucleic acids having base sequences hybridizable with the first nucleic acids and being fluorescence-labeled;

(3) forming a reaction system in which a first nucleic acid and a second nucleic acid can form a hybrid in each of the immobilizing regions of the substrate;

(4) forming and dissociating the hybrid in each of the reaction systems by changing a temperature of each of the reaction systems;

(5) measuring an intensity of fluorescence, which is derived from each of the fluorescence-labeled second nucleic acids and is generated or disappears in accordance with formation or dissociation of the hybrid, by focusing an optical detection system of a detection device on the surface of the substrate; and

(6) determining a melting temperature of each of the hybrids on the substrate based on a profile of the intensity of fluorescence measured by the detection device.

According to the present invention, there is provided an apparatus for measuring a melting temperature of a double-stranded nucleic acid, comprising:

sample holding means for holding a sample having a reaction system comprising a nucleic acid chip having an immobilizing region for a first nucleic acid on a substrate, and a liquid present in contact with the immobilizing region and containing a second nucleic acid having a base sequence hybridizable with the first nucleic acid and being fluorescence-labeled;

temperature controlling means for controlling a temperature of the reaction system;

temperature detecting means for detecting the temperature of the reaction system;

temperature recording means for recording a change in the temperature of the reaction system detected by the temperature detecting means;

a detection device for detecting an intensity of fluorescence derived from the fluorescence-labeled second nucleic acid on a surface of the substrate of the nucleic acid chip held by the sample holding means; and

fluorescence intensity recording means for recording the intensity of fluorescence detected on the surface of the substrate by the detection device,

wherein a melting temperature of a hybrid formed of the first and second nucleic acids is determined based on a profile showing a change in the intensity of fluorescence on the surface of the substrate, derived from the fluorescence-labeled second nucleic acid depending upon the change in the temperature of the reaction system.

According to the present invention, there is provided a system for measuring a melting temperature of a double-stranded nucleic acid, comprising:

at least one of a reagent and a tool for preparing a sample having a reaction system comprising a nucleic acid chip having an immobilizing region for a first nucleic acid on a substrate, and a liquid present in contact with the immobilizing region and containing a second nucleic acid having a base sequence hybridizable with the first nucleic acid and being fluorescence-labeled; and

the apparatus having the aforementioned structure.

According to the present invention, it is possible to measure the melting temperature of a double-stranded nucleic acid bound to a substrate and the melting temperatures of double-stranded nucleic acids hybridized with corresponding probes of a nucleic acid chip.

Other features and advantages of the present invention will be apparent from the following description taken in conjunction with the accompanying drawings, in which like reference characters designate the same or similar parts throughout the figures thereof.

### BRIEF DESCRIPTION OF THE DRAWINGS

FIG. 1 is a schematic view of a melting temperature measurement apparatus;

FIG. 2 is a view for explaining a temperature control mechanism of the melting temperature measuring apparatus;

FIG. 3 is a sectional view of a nucleic acid chip in which a reaction system for forming or dissociating a nucleic acid hybrid formed on a substrate; and

FIG. 4 is a view for explaining the constitution of an optical system that the melting temperature measuring apparatus has.

### DESCRIPTION OF THE PREFERRED EMBODIMENTS

Preferred embodiments of the present invention will now be described in detail in accordance with the accompanying drawings.

In the method for measuring the melting temperature of a double-stranded nucleic acid according to the present invention, a nucleic acid chip is used in which one of the single-stranded nucleic acids (hereinafter referred to as a “first nucleic acid” and a “second nucleic acid”) constituting the double-stranded nucleic acid (hybrid) is immobilized on a substrate. The first nucleic acid may be immobilized on a substrate in accordance with any customary method as long as a stable immobilization state of the nucleic acid can be obtained in contact with a hybridization reaction solution and even if the temperature is changed for measuring the melting temperature.

A plurality of immobilizing regions are separately provided for immobilizing different types of first nucleic acids. In this manner, the melting temperatures of different types of hybrids (since the types of first nucleic acids differ) can be separately measured.

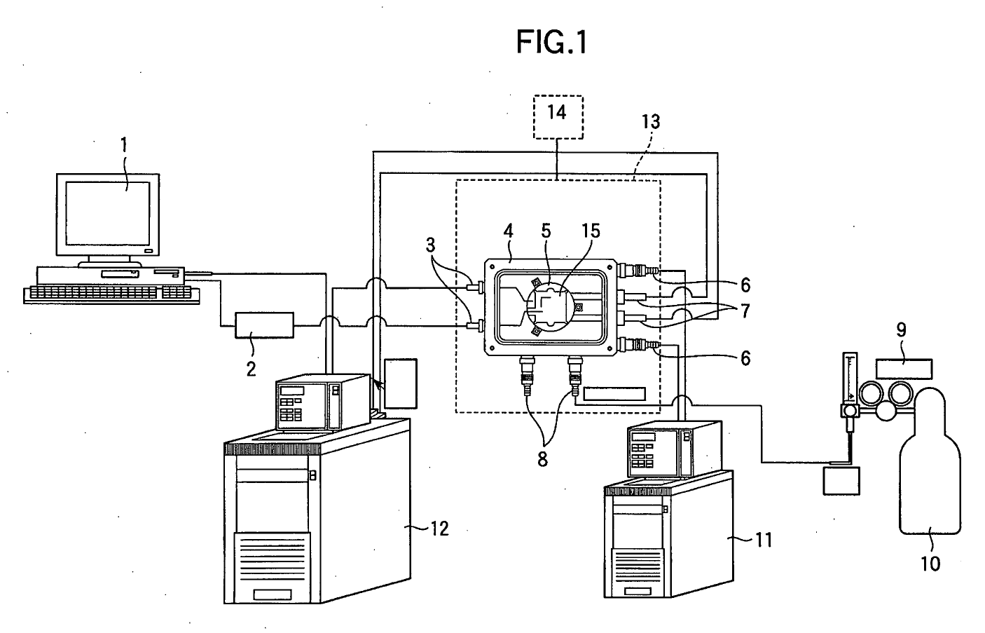

FIG. 1 shows a melting temperature measuring apparatus for a double-stranded nucleic acid according to the present invention. The apparatus is constituted by at least

a chamber 4 in which a reaction system (not shown) for forming or dissociating a hybrid formed of the first and second nucleic acids,

temperature detecting means for detecting the temperature of a chamber 4,

temperature controlling means for controlling the temperature of the chamber 4,

a detection device 13 for detecting fluorescence emitted from a substrate surface of a nucleic acid chip (not shown) present in the reaction system formed in the chamber 4, and

position controlling means for controlling a sample stage 5 arranged in the chamber 4.

In the figure, reference numeral 6 shows an inlet/outlet of pure water for cooling the chamber, 7 an inlet/outlet of constant-temperature water for a sample-stage, 8 an inlet/outlet of nitrogen purge gas, and 9 a valve for reducing pressure.

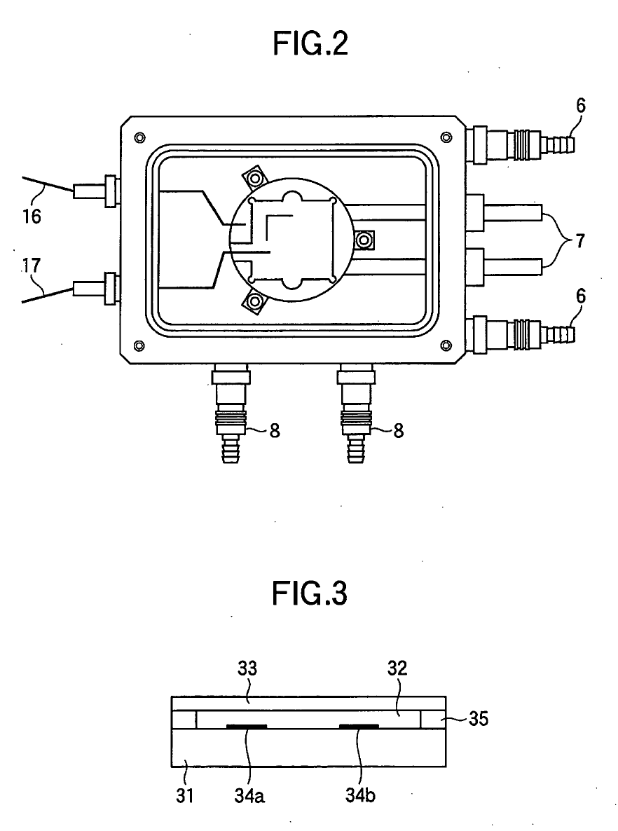

The temperature detecting means is constituted by a pair of temperature sensors (such as resistance temperature sensors) 3, a data loader for loading data of temperature 2, and computer 1. One of the pair of temperature sensors 3 (a sensor 16 of FIG. 2) is arranged at a position at which the temperature of the sample stage 5 can be detected and the other (a sensor 17 of FIG. 2) is arranged at a position at which the temperature of a sample (for example, the temperature of a top plate covering a sample shown in FIG. 3) can be detected. The temperature is measured and data of the temperature is stored by the computer 1 and data loader 2. The computer 1 stores data of the temperature detected by the temperature sensors and a temperature control program for controlling temperature of the reaction region (represented by reference numeral 32 in FIG. 3) within the nucleic acid chip for measuring a melting temperature. The temperature of the chamber 4 can be controlled by, for example, a circulator 12. If necessary, the chamber 4 may be cooled by the circulator 11.

In the apparatus, the temperatures of individual reaction systems in the chamber are integrally controlled. If necessary, heating means may be locally provided for separately controlling the temperatures of individual reaction systems.

A fluorescent signal emitted from the surface of a nucleic acid chip 15 held on the sample stage 5 is detected by fluorescence detecting means (not shown) such as a photosensor, and sent to a computer 14 in the form of data and stored therein. The data input in the computer 14 is compared with the data of the temperature of the sample previously stored in the computer 1 to determine the melting temperature. The comparison operation can be performed in accordance with a predetermined program by connecting the computer 1 to the computer 14. Furthermore, the computers 1 and 14 may be integrated in a single computer.

The position of the stage 5 can be controlled with respect to the detection device 13. The position of the stage 5 can be controlled by movably forming at least one of the stage 5 and the detection device 13 and in accordance with the program stored in the computer 14.

On the other hand, the top of the chamber 4 has a light permeable window for introducing excitation light from the detection device 13 and taking out fluorescence. To prevent dew condensation on a window, the chamber 4 is purged with nitrogen gas supplied from a nitrogen gas cylinder 10.

FIG. 3 shows a schematic view of a nucleic acid chip in which different types of first nucleic acids 34a and 34b are respectively immobilized in immobilizing regions partitioned on the surface of a substrate 31.

A second nucleic acid(s) capable of forming a hybrid with a first nucleic acid(s) is contained in the hybridization reaction solution 32, and arranged in the immobilizing region(s) of the first nucleic acid(s) on the substrate 31 in contact with the first nucleic acid(s). In the example shown in FIG. 3, the reaction solution 32 is kept in a structure regulated by a frame 35 and the top plate 33 formed in a predetermined position on the substrate. The reaction solution 32 contains a reaction system required for hybridization of the first and second nucleic acids. Any reaction solution may be used as the reaction solution 32 as long as it has a composition permitting to form a hybrid(s) under predetermined temperature conditions for measuring a melting temperature. The reaction solution may be appropriately chosen from hybridization reaction solutions generally used.

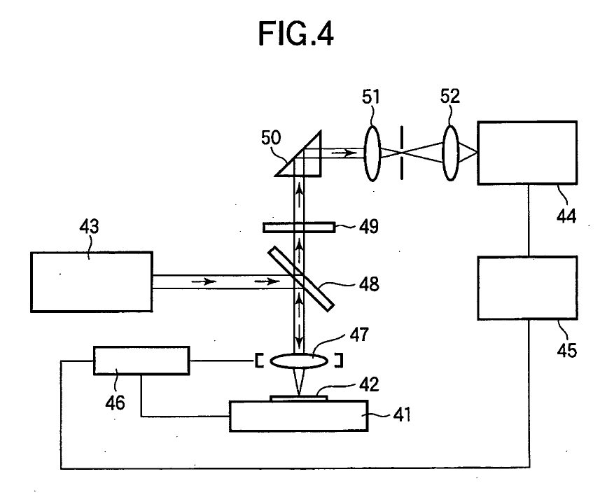

Next, a sample having a reaction system on the surface of the nucleic acid chip is immobilized on a stage serving as sample-holding means. As the detection device, it is preferred to use a system constituting a confocal fluorescence microscope. FIG. 4 schematically shows the constitution of a melting temperature measuring apparatus having a confocal fluorescence microscope. The melting temperature measuring system is constituted by at least

a light source 43 for exciting a fluorescent label,

an optical system (including a dichroic mirror 48 and an objective lens 47) for applying a spot of light from the light source onto the immobilizing region (not shown) of the first nucleic acid(s) on the surface of the substrate 31 (shown in FIG. 3),

another optical system (including, objective lens 47, the dichroic mirror 48, filter 49, prism 50, lenses 51 and 52) for guiding fluorescence emitted from the surface of the substrate to fluorescence detecting means,

fluorescence detecting means 44,

a computer 45 having a recorder for recording data of fluorescence intensity detected by the fluorescence detecting means, and

focus controlling means 46.

Note that the detailed structures of the chamber and sample shown in FIGS. 1 to 3 are omitted in FIG. 4. Note that the fluorescence detecting means, recorder and focus controlling means may be constituted by the computer 14 (for example, personal computer) shown in FIG. 1.

A hybrid is formed or dissociated on a substrate surface of a sample 42 while controlling the temperature of the sample 42 by the temperature controlling means shown in FIGS. 1 and 2. As the reaction system of the sample 42, any type of reaction solution may be used as long as it can mediate reversible hybridization between the first nucleic acid (immobilized on the substrate) and the second nucleic acid depending upon temperature change.

The melting temperature of a hybrid formed on the substrate surface can be determined based on the profile of fluorescence intensity (which is obtained by applying excitation light from the light source 43, focusing on the substrate surface like a spot, and detecting the fluorescence emitted from the sample 42 on the substrate surface) and the temperature change in the sample 42.

The profile of fluorescence intensity can be prepared by recording the change in measured fluorescence intensity with respect to the temperature measured in the temperature measuring means, or when the temperature change in the reaction system is sufficiently linear with respect to the measurement time, with respect to the time necessary for measurement.

The position of the objective lens 47 with respect to the stage 41 can be automatically controlled by focus controlling means 46 in accordance with a program stored in the computer 44. The stage 41 may be moved in the X, Y and/or Z axis. As a method of focusing the optical detection system on the substrate surface of the nucleic acid chip to capture the fluorescence emitted from the fluorescent substance excited, the following methods are preferably used.

(1) A method of automatically controlling the distance between the objective lens 47 and the substrate surface so as to detect the reflection position (on the substrate surface) of the light beam, which is applied from the light source 43 to the substrate surface of the sample 42.

(2) A method of automatically changing the distance between the objective lens 47 and the substrate surface so as to determine a point at which fluorescence emitted from the second nucleic acid or from a fluorescent marker previously bound to the substrate surface, is obtained with maximum intensity, as a focal point.

(3) A method of continuously changing the distance between the objective lens 47 and the substrate surface to continuously obtain the fluorescence intensity emitted from the second nucleic acid or from the fluorescent marker previously bound to the substrate surface, and thereafter determining the maximum intensity of fluorescence derived from the second nucleic acid as the fluorescence intensity at a focal point.

In the method (3), for example, fluorescence intensities continuously obtained are stored in a memory. The fluorescence intensity at the focal point may be determined by selecting the maximum value from the data of the fluorescence intensities stored in the memory.

The measurement accuracy of a melting temperature may be improved by previously binding the same fluorescent substance as that labels the second nucleic acid to the substrate surface of the sample 42 as a marker, obtaining the intensities of fluorescence emitted from the second nucleic acid and from the marker simultaneously, correcting the profile of the intensity of fluorescence emitted from the second nucleic acid based on the profile of the intensity of fluorescence from the marker.

According to the present invention, since the melting temperature is measured by use of not absorbance but fluorescence, the melting temperature of a long-chain nucleic acid, which is not measured by a conventional technique, can be measured. Besides this, the melting temperature in the case of solid phase hybridization can be measured. In addition, since fluorescence of the substrate surface is detected, it is possible to overcome the problem of the DASH method: a second fluorescence-labeled nucleic acid cannot be used, and the problem caused by an intercalating dye.

In the nucleic acid chip of the sample 42, a single type of first nucleic acid may be immobilized. Alternatively, a plurality of types of first nucleic acids may be immobilized in mutually discrete regions, as shown in FIG. 3. When immobilizing regions for a plurality of types of probes are formed in the substrate surface and nucleic acids hybridizable with individual probes are contained in a reaction system, the melting temperatures of hybrids with the plurality type of probes can be simultaneously measured. In this case, fluorescence intensity of each of immobilizing regions (nucleic acid spots, nucleic acid dots) on the surface of the nucleic acid chip is preferably taken as an image altogether. By recording fluorescence intensity varied with temperature change in the nucleic acid chip in this manner, a melting temperature can be determined.

According to an embodiment of immobilizing a plurality of types of first nucleic acids, a melting temperature can be determined by measuring fluorescence on the substrate surface (solid surface) constituting a nucleic acid chip. In addition to this advantage, the melting temperatures of a plurality of types of first nucleic acids (probes) and a plurality of types of second nucleic acids hybridizing with them can be simultaneously measured. As a result, objects of measuring melting temperatures of many nucleic acids in a liquid phase can be attained.

An apparatus for measuring a melting temperature can be formed by use of at least one of reagents and tools for forming a sample and the system having the aforementioned constitution. More specifically, such an apparatus may be provided by using one or more elements selected from the reagents and tools including a first nucleic acid, second nucleic acid, substrate, fluorescent marker, hybridization reaction solution (buffers), and a cover and a frame for forming a sample, in combination with the apparatus.

The present invention will be further described more specifically by way of Examples.

### EXAMPLE 1

Example for measuring a melting temperature by use of reflection of a light beam externally applied to obtain focus:

(1) Preparation of Nucleic Acid Chip

A nucleic acid chip was prepared by the following method in accordance with the method described in Japanese Patent Application Laid-Open No. H11-187900

(1-1) Washing of a Substrate

A synthesized quartz substrate of 25.4 mm×25.4 mm×1 mm was placed in a rack and soaked overnight in a 10% ultrasonic cleaner (BRANSON: GPIII) diluted with pure water. Thereafter, the substrate was washed with ultrasonic wave in the cleaner for 20 minutes and washed with water to remove the cleaner. After rinsed with pure water, the substrate was further treated with ultrasonic wave for 20 minutes in a container containing pure water. The substrate was then soaked for 10 minutes in a 1N aqueous sodium hydroxide solution previously heated to 80° C. Subsequently, the substrate was washed with water and then with pure water, and directly subjected to the next step.

(1-2) Surface Treatment

A 1 wt % aqueous solution containing a silane coupling agent having an amino group bound thereto, and N-β-(aminoethyl)-γ-aminopropyltrimethoxysilane KBM603 (Shin-Etsu Chemical Co., Ltd.) was stirred at room temperature for two hours to hydrolyze an intramolecular methoxy group in the silane compound. Subsequently, the substrate obtained in the step (1-1) above was soaked at room temperature for about one hour, washed with pure water and dried by blowing nitrogen gas to both surfaces of the substrate. Then, the substrate was baked in an oven heated to 120° C. for one hour to introduce the amino group into the surface of the substrate.

Next, 2.7 mg of N-maleimido-caproyloxy-succinimide (manufactured by DOJINDO LABORATORIES, hereinafter referred to as “EMCS”) was dissolved in a solution mixture containing dimethylsulfoxide (DMSO)/ethanol (1:1) to a concentration of 0.3 mg/ml. The substrate treated with the silane-coupling agent was soaked in the EMCS solution at room temperature for 2 hours to react the amino group carried on the substrate surface by silane coupling treatment with a succinimide group derived from the EMCS solution. In this stage, a maleimide group derived from the EMCS was present on the substrate surface. This treatment was also performed to prevent adhesion of a nucleic acid onto the surface of the substrate. The substrate picked up form the EMCS solution was successively washed with the solvent mixture of DMSO/ethanol and ethanol, and subsequently dried by blowing nitrogen gas.

(1-3) Synthesis of Probe DNA (First Nucleic Acid)

Single-stranded nucleic acids represented by SEQ ID NOs: 1 to 6 (having 20, 25, 30, 35, 40 and 45 dT units, respectively) were synthesized by ordering the synthesis to a supplier of synthesized DNA (BEX CO., LTD.). Note that a thiol modifier (Glen Research) was used in synthesizing a single-stranded DNA of SEQ ID NO: 1 to introduce a thiol (SH) group into the 5′ end. Deprotection and recovery of DNA were performed in accordance with customary methods and purification was performed by high performance liquid chromatography (HPLC). A series of synthesis steps was all performed by the supplier for synthesized DNA.

SEQ ID NO: 1

5′ HS-(CH₂)₆-O-PO₂-O-TTTTTTTTTT TTTTTTTTTT 3′

SEQ ID NO: 2

5′ HS-(CH₂)₆-O-PO₂-O-TTTTTTTTTT TTTTTTTTTT
TTTTTT 3′

SEQ ID NO: 3

5′ HS-(CH₂)₆-O-PO₂-O-TTTTTTTTTT TTTTTTTTTT
TTTTTTTTTT 3′

SEQ ID NO: 4

5′ HS-(CH₂)₆-O-PO₂-O-TTTTTTTTTT TTTTTTTTTT
TTTTTTTTTT TTTTT 3′

SEQ ID NO: 5

5′ HS-(CH₂)₆-O-PO₂-O-TTTTTTTTTT TTTTTTTTTT
TTTTTTTTTT TTTTTTTTTT 3′

SEQ ID NO: 6

5′ HS-(CH₂)₆-O-PO₂-O-TTTTTTTTTT TTTTTTTTTT
TTTTTTTTTT TTTTTTTTTT TTTTT 3′

(1-4) Synthesis of Fluorescent Marker

A fluorescent marker represented by SEQ ID NO: 7 was synthesized by ordering the supplier for synthesized DNA (BEX CO., LTD.). Note that phosphoroamidite was used to introduce a Cy3 marker to the 5′ end and a thiol modifier (Glen Research) was used to introduce a thiol (SH) group into the 3′ end. Deprotection and recovery of DNA were performed in accordance with customary methods and purification was performed by HPLC. A series of synthesis process were all performed by the supplier for synthesized DNA.

SEQ ID NO: 7

5′ Cy3-(CH₂)₆-O-PO₂-O-CCCCCCCCCC CCCCCCCCCC
CCCCC-O-PO₂-O-(CH₂)₆-SH 3′

(1-5) Ejection of DNA by Thermal Inkjet Printer and Binding of DNA to Substrate

The single-stranded DNAs represented by SEQ ID NOs. 1 to 6 were dissolved in a solution containing glycerin (7.5 wt %), urea (7.5 wt %), thioglycol (7.5 wt %), and acetylene alcohol (1 wt %) (trade name: Acetylenol EH, manufactured by Kawaken Fine Chemicals Co., Ltd.) at a concentration of 8 μM. In the same manner, the fluorescent marker represented by SEQ ID NO. 7 was dissolved in the solution to an absorbance of 0.1. A printer head BC-50 (Canon Inc.) of a bubble jet printer, BJF-850 (Canon Inc.) employing bubble jet method, a type of thermal jet method, was modified so as to eject a solution of several hundreds of μl. The head was installed in an ejection spotter, which was previously modified for spraying a solution onto the quartz substrate as mentioned above. The 6 types of DNA solutions (each several hundreds of μl) were injected into modified tanks of the 6 heads and spotted to the substrate, which was previously treated by EMCS and set in the ejection spotter. Note that the ejection amount of DNA solution in spotting was 4 pl/droplet. The DNA solution was spotted within the area of 10 mm×10 mm at the center of the substrate at intervals of 200 dpi (dot per inch), that is, at a pitch of 127 μM. In this condition, the diameters of dots thus spotted were about 50 μm. Spots were arranged in a 5×4 matrix by arranging the spots of 6 types of DNA at the center and the spots of the marker around the DNA spots. In this example, 10 of this pattern were prepared by spotting.

After completion of spotting, the substrate was placed in a moisture chamber and allowed to stand still for 30 minutes to react the maleimide group on the glass surface with the nucleic acid probe and the terminal thiol group of the marker. Subsequently, the substrate was washed in pure water and stored in pure water. The substrate was then subjected to measurement of a melting temperature.

(2) Preparation of Hybridization Reaction Field

On the upper surface (having probes bound thereto) of the nucleic acid chip prepared in the step (1), a frame (internal dimensions: 15×16 mm, width: 8 mm, thickness: 0.25 mm, trade name: hybridization frame, manufactured by Nippon Genetics Co., Ltd.) having an adhesive agent applied on both surfaces was attached. A cover glass of 22×22 mm (thickness: 0.17 mm) having holes at both ends was attached on the frame. In this manner, a sample having a reaction field enclosed therein was prepared. The hybridization solution having the following composition (65 μl) was added dropwise through an end of the cover glass and both end-holes of the cover glass were sealed with polyimide tape. Note that SEQ ID NO: 8 shown below represents a common complementary nucleic acid (second nucleic acid) to the aforementioned 6 types of probes and labeled with fluorescent dye Cy3.

Hybridization Solution:

6×SSPE, 10% formamide

50 nM Cy3-labeled dA45 (SEQ ID NO: 8 below)

SEQ ID NO: 8

5′ Cy3-(CH₂)₆-O-PO₂-O-AAAAAAAAAA AAAAAAAAAA
AAAAAAAAAA AAAAAAAAAA AAAAA 3′

(3) Measurement of Melting Temperature

The sample prepared in the step (2) was set at the stage (equipped with a temperature controller) of the melting temperature measurement apparatus 5 shown in FIG. 1. The temperature controller was set such that the temperature was increased or decreased at a rate of 0.5° C./min within the range of 25° C. to 80° C. In this manner, the temperature of the cover glass (hybridization solution) was controlled and monitored by a resistance temperature sensor set at the surface of the cover glass every one minute and taken as data. The fluorescence intensity was monitored by setting a 20× fluorescence observation objective lens (Fluor) and a Cy3 fluorescence observation filter block (No. 20) to LSM510. The nucleic acid chip was irradiated with a red semiconductor laser from a light source arranged outside the microscope and reflection light thereof was detected by a photosensor arranged outside the microscope. With reference to the position of the photosensor at which the reflection light was successfully detected, the height of the stage of the microscope was controlled to bring the sample into focus. Then, the temperature of the sample was successively increased and decreased. The temperature captured by the resistance temperature sensor set at the surface of the cover glass and an average fluorescence intensity per pixel of each spot were taken as data every one minute.

Based on the data, the melting temperatures of the probes and the complementary nucleic acid thereto were obtained. The results are shown in Table 1.

TABLE 1

| Probe | dT20 | dT25 | dT30 | dT35 | dT40 | dT45 |
|---|---|---|---|---|---|---|
| Melting temperature (° C.) | 38.3 | 43.4 | 46.8 | 49.2 | 51.0 | 52.4 |

From the Table 1, it is demonstrated that the melting temperatures of nucleic acid probes and the complementary nucleic acid on a nucleic acid chip can be measured by an apparatus or a method for measuring the melting temperature of a nucleic acid according to the present invention.

### EXAMPLE 2

Example of measuring a melting temperature by focusing a sample by automatically changing the distance between an objective lens of the optical detection system and the substrate surface:

The sample obtained in Example 1 was set at the stage (equipped with a temperature controller) of the melting temperature measurement apparatus 5 shown in FIG. 1. The temperature controller was set such that the temperature was increased or decreased at a rate of 0.5° C./min within the range of 25° C. to 80° C. In this manner, the temperature of the cover glass (hybridization solution) was controlled and monitored by a resistance temperature sensor set at the surface of the cover glass every one minute. The fluorescence intensity was monitored by setting a 20× fluorescence observation objective lens (Fluor) and a Cy3 fluorescence observation filter block (No. 20) to LSM510. While the stage of the confocal microscope was moved in the height direction (Z-axis), the temperature of the sample was successively increased and decreased to detect the position at which the intensity of fluorescence derived from the second nucleic acid or from the fluorescence marker on the nucleic acid chip exhibits a maximum value. An image was taken at the position at which the maximum fluorescence intensity was obtained. The temperature captured by the resistance temperature sensor set at the surface of the cover glass and an average fluorescence intensity per pixel of each spot were taken as data every one minute.

Based on the data, the melting temperatures of the probes and the complementary nucleic acid thereto were obtained. The results are the same as shown in Table 1.

### EXAMPLE 3

Example of measuring a melting temperature by taking pictures of images at every positions while gradually changing the distance between an objective lens of the optical detection system and the substrate surface, selecting the image exhibiting maximum intensity of fluorescence derived from a second nucleic acid or a fluorescence marker, and employing the maximum intensity as the fluorescence intensity at a focal point:

The sample obtained in Example 1 was set at the stage 41 (equipped with a temperature controller) of the melting temperature measurement apparatus 5 shown in FIG. 1. The temperature controller was set such that the temperature was increased or decreased at a rate of 0.5° C./min within the range of 25° C. to 80° C. In this manner, the temperature of the cover glass (hybridization solution) was controlled and monitored by a resistance temperature sensor set at the surface of the cover glass every one minute. The fluorescence intensity was monitored by setting a 20× fluorescence observation objective lens (Fluor) and a Cy3 fluorescence observation filter block (No. 20) to LSM510. Images were automatically taken while moving the Z axis of the stage of the microscope (confocal microscope) at a rate of 1 μm/minute by use of “Time Series” software installed in the microscope. Of the image data thus taken, an image datum taken at the height at which the intensity of the fluorescent marker exhibited a maximum value, is selected. Then an average fluorescence intensity per pixel of each spot was obtained. The fluorescence intensity of the fluorescent marker varying depending upon the temperature was corrected with respect to the average fluorescence intensity and then plotted against the temperature.

Based on the data thus taken, the melting temperatures of the probes and the complementary nucleic acids were obtained, the results are the same as shown in Table 1.

### EXAMPLE 4

Example of measuring a melting temperature when the intensity of fluorescence derived from the second nucleic acid is corrected by that derived from the fluorescent marker on the substrate:

The sample obtained in Example 1 was set at the stage 41 (equipped with a temperature controller) of the melting temperature measurement apparatus 5 shown in FIG. 1. The temperature controller was set such that the temperature was increased or decreased at a rate of 0.5° C./min within the range of 25° C. to 80° C. In this manner, the temperature of the cover glass (hybridization solution) was controlled and monitored by a resistance temperature sensor set at the surface of the cover glass every one minute. The fluorescence intensity was monitored by setting a 20× fluorescence observation objective lens (Fluor) and a Cy3 fluorescence observation filter block (No. 20) to LSM510. The nucleic acid chip was irradiated with a red semiconductor laser from a light source arranged outside the microscope and reflection light thereof was detected by a photosensor arranged outside the microscope. With reference to the position of the photosensor at which the reflection light was successfully detected, the height of the stage of the microscope was controlled to bring the sample into focus. Then, the temperature of the sample was successively increased and decreased. The temperature captured by the resistance temperature sensor set at the surface of the cover glass and an average fluorescence intensity per pixel of each spot were taken as data every one minute.

The intensity of fluorescence derived from the second nucleic acid thus taken was divided by the intensity of fluorescence derived from the fluorescence marker at the same time point. Then, the primary differntial of the division was obtained and plotted on the longitudinal axis. On the lateral axis, temperature was plotted. In this way, the melting temperature was obtained. The results are the same as shown in Table 1.

The present invention is not limited to the above embodiments and various changes and modifications can be made within the spirit and scope of the present invention. Therefore to apprise the public of the scope of the present invention, the following claims are made.

This application claims priority from Japanese Patent Application No. 2005-189840 filed on Jun. 29, 2005, which is hereby incorporated by reference herein.

What is claimed is:

1. A method of measuring a melting temperature of a double-stranded nucleic acid comprising the steps of:
(1) supplying a second nucleic acid having a base sequence hybridizable with a first nucleic acid to a substrate having the first nucleic acid immobilized thereon to form a reaction system;
(2) monitoring a change in a reaction by changing a temperature of the reaction system; and
(3) determining a melting temperature of a hybrid formed on the substrate based on a profile of the reaction monitored.

2. The method according to claim 1, wherein, in the step of monitoring a change in a reaction, the second nucleic acid is fluorescence-labeled, and an intensity of fluorescence derived from the fluorescence-labeled second nucleic acid, which is generated or disappears on a surface of the substrate in accordance with formation or dissociation of the hybrid, is measured by focusing an optical detection system of a detection device on the surface of the substrate.

3. The method according to claim 2, wherein the optical detection system is focused on the surface of the substrate by externally irradiating the surface of the substrate with a light beam, and automatically adjusting a distance between an objective lens of the optical detection system and the surface of the substrate so as to detect a reflection position of the light beam on the surface of the substrate.

4. The method according to claim 2, wherein the optical detection system is focused on the surface of the substrate by automatically changing a distance between an objective lens of the optical detection system and the surface of the substrate to obtain, as a focal point, a position at which fluorescence derived from the second nucleic acid or fluorescence derived from a fluorescent marker previously bound to the surface of the substrate exhibits a maximum fluorescence intensity.

5. The method according to claim 2, wherein the optical detection system is focused on the surface of the substrate by continuously changing a distance between an objective lens of the optical detection system and the surface of the substrate to continuously obtain intensities of fluorescence derived from the second nucleic acid or fluorescence derived from a fluorescent marker previously bound to the surface of the substrate, and determining a point at which a maximum fluorescence intensity is obtained as a focal point.

6. The method according to claim 2, wherein the detection device is a confocal fluorescence microscope.

7. The method according to claim 2, wherein the same fluorescent substance as that labels the second nucleic acid is previously bound to the surface of the substrate as a marker, the intensity of fluorescence derived from the second nucleic acid and an intensity of fluorescence derived from the marker are simultaneously obtained, and a profile of the intensity of fluorescence derived from the second nucleic acid is corrected by a profile of the intensity of fluorescence derived from the marker.

8. A method of measuring a melting temperature of a double-stranded nucleic acid comprising the steps of:
(1) preparing a nucleic acid chip in which a plurality of types of first nucleic acids are separately immobilized on a surface of a substrate partitioned into immobilizing regions;
(2) preparing a plurality of types of second nucleic acids having base sequences hybridizable with the first nucleic acids and being fluorescence-labeled;
(3) forming a reaction system in which a first nucleic acid and a second nucleic acid can form a hybrid in each of the immobilizing regions of the substrate;
(4) forming and dissociating the hybrid in each of the reaction systems by changing a temperature of each of the reaction systems;
(5) measuring an intensity of fluorescence, which is derived from each of the fluorescence-labeled second nucleic acids and is generated or disappears in accordance with formation or dissociation of the hybrid, by focusing an optical detection system of a detection device on the surface of the substrate; and
(6) determining a melting temperature of each of the hybrids on the substrate based on a profile of the intensity of fluorescence measured by the detection device.

9. The method according to claim 8, wherein the optical detection system is focused on the surface of the substrate by externally irradiating the surface of the substrate with a light beam and automatically adjusting a distance between an objective lens of the optical detection system and the surface of the substrate so as to detect a reflection position of the light beam on the surface of the substrate.

10. The method according to claim 8, wherein the optical detection system is focused on the surface of the substrate by automatically changing a distance between an objective lens of the optical detection system and the surface of the substrate to obtain, as a focal point, a position at which fluorescence derived from the second nucleic acid or fluorescence derived from a fluorescent marker previously bound to the surface of the substrate exhibits a maximum fluorescence intensity.

11. The method according to claim 8, wherein the optical detection system is focused on the surface of the substrate by continuously changing a distance between an objective lens of the optical detection system and the surface of the substrate to continuously obtain intensities of fluorescence derived from the second nucleic acid or fluorescence derived from a fluorescent marker previously bound to the surface of the substrate, and determining a point at which a maximum fluorescence intensity is obtained as a focal point.

12. The method according to claim 8, wherein the detection device is a confocal fluorescence microscope.

13. The method according to claim 8, wherein the same fluorescent substance as that labels the second nucleic acid is previously bound to the surface of the substrate as a marker, the intensity of fluorescence derived from the second nucleic acid and an intensity of fluorescence derived from the marker are simultaneously obtained, and a profile of the intensity of fluorescence derived from the second nucleic acid is corrected by a profile of the intensity of fluorescence derived from the marker.

14. An apparatus for measuring a melting temperature of a double-stranded nucleic acid, comprising:
sample holding means for holding a sample having a reaction system comprising a nucleic acid chip having an immobilizing region for a first nucleic acid on a substrate, and a liquid present in contact with the immobilizing region and containing a second nucleic acid having a base sequence hybridizable with the first nucleic acid and being fluorescence-labeled;
temperature controlling means for controlling a temperature of the reaction system;
temperature detecting means for detecting the temperature of the reaction system;
temperature recording means for recording a change in the temperature of the reaction system detected by the temperature detecting means;
a detection device for detecting an intensity of fluorescence derived from the fluorescence-labeled second nucleic acid on a surface of the substrate of the nucleic acid chip held by the sample holding means; and
fluorescence intensity recording means for recording the intensity of fluorescence detected on the surface of the substrate by the detection device,
wherein a melting temperature of a hybrid formed of the first and second nucleic acids is determined based on a profile showing a change in the intensity of fluorescence on the surface of the substrate, derived from the fluorescence-labeled second nucleic acid depending upon the change in the temperature of the reaction system.

15. The apparatus for measuring a melting temperature according to claim 14, further comprising focus controlling means for focusing an optical detection system of the detection device on the surface of the substrate.

16. The apparatus for measuring a melting temperature according to claim 15, wherein the focus controlling means has means for externally irradiating the surface of the substrate with a light beam, and means for automatically controlling a position of an objective lens of the optical detection system with respect to the surface of the substrate at a reflection point of the light beam on the surface of the substrate.

17. The apparatus for measuring a melting temperature according to claim 16, wherein the focus controlling means has means for automatically controlling a distance between the objective lens of the optical detection system and the surface of the substrate so as to obtain a maximum intensity of fluorescence derived from the second nucleic acid obtained on the surface of the substrate or fluorescence derived from a fluorescent marker previously bound to the surface of the substrate.

18. The apparatus for measuring a melting temperature according to claim 16, wherein the focus controlling means has means for continuously obtaining intensities of fluorescence derived from the second nucleic acid on the surface of the substrate or fluorescence derived from a fluorescent marker previously bound to the surface of the substrate while continuously changing a distance between the objective lens of the optical detection system and the surface of the substrate, and means for selecting a maximum intensity of fluorescence derived from the second nucleic acid or the fluorescent marker.

19. The apparatus for measuring a melting temperature according to claim 16, wherein the detection device is a confocal fluorescence microscope having the focus controlling means.

20. The apparatus for measuring a melting temperature according to claim 15, wherein the substrate used has a marker of the same fluorescent substance as that labels the second nucleic acid, the apparatus further comprises marker fluorescence detection means for detecting a change in intensity of fluorescence derived from the marker, and a profile of the intensity of fluorescence derived from the marker is used for correcting a profile of the intensity of fluorescence derived from the fluorescence-labeled second nucleic acid.

21. The apparatus for measuring a melting temperature according to claim 15, wherein a plurality of immobilizing regions are provided on the surface of the substrate.

22. A system for measuring a melting temperature of a double-stranded nucleic acid, comprising:
at least one of a reagent and a tool for preparing a sample having a reaction system comprising a nucleic acid chip having an immobilizing region for a first nucleic acid on a substrate, and a liquid present in contact with the immobilizing region and containing a second nucleic acid having a base sequence hybridizable with the first nucleic acid and being fluorescence-labeled; and
the apparatus according to claim 14.

23. The system for measuring a melting temperature according to claim 22, wherein a plurality of immobilizing regions are provided on a surface of the substrate.
